(19) United States
(12) Patent Application Publication
(10) Pub. No.: US 2003/0109804 A1
(43) Pub. Date: Jun. 12, 2003

(54) Cell collection apparatus

(76) Inventors: Robert D. Auerbach, Madison, CT (US); Peter Auerbach, Groton, CT (US); Michael Barenboym, Ashland, MA (US)

Correspondence Address: Neal L. Rosenberg, Esq., AMSTER, ROTHSTEIN & EBENSTEIN, 90 Park Avenue, New York, NY, 10016, US

(21) Appl. No.: 10/014,119

(22) Filed: Dec. 11, 2001

(51) Int. Cl. A61B 10/00

(52) U.S. Cl. 600/569

(57) ABSTRACT

A collection container for containing a solution useful for retrieval of cell samples both from a spatula having a collector with ectocervical cell samples thereon and from a cytology brush having bristles with endocervical cell samples thereon.

### BACKGROUND OF THE INVENTION

The present invention relates to a collection apparatus for containing a solution useful for retrieval of cell samples, and more particularly, to such apparatus for retrieving cervical cell samples both from a spatula having a collector and from a cytology brush having bristles.

U.S. Pat. No. 5,422,273 discloses a cell collection apparatus which utilizes a combination of fins for agitating the bristles of a cytology brush within a fixative solution, thereby permitting retrieval of the cell samples from the cytology brush. While the apparatus is said to be useful in the collection of cell samples from cytology brushes having different configurations, the disclosed apparatus has utility only in the collection of endocervical cells—that is, the cells removed from within the interior of the cervix by a cytology brush. Current medical standards require that the Papanicolaou (“Pap”) test be performed on a collection of both endocervical cell samples and ectocervical cell samples. While endocervical cells are found in the interior of the cervix and removed therefrom by a cytology brush having on a leading end thereof bristles which are inserted into the interior of the cervix, endocervical cells are found on the exterior of the cervix and are removed therefrom with a spatula having on a leading end thereof a collector (rather than bristles) for scraping endocervical cells from the exterior of the cervix.

For a variety of different reasons including inter alia the central location of the cell removal member within the container of the disclosed apparatus, the conically shaped closed end of the disclosed apparatus, and the presence of an alignment member at the open end of the container of the disclosed apparatus, the disclosed apparatus is simply not suited for the removal of ectocervical cell samples from the spatula typically used to collect such ectocervical cell samples from the exterior of the cervix. The aforementioned features of the disclosed apparatus limit the ability of the spatula to be moved within the solution and, in particular, to be struck against an abutment surface with sufficient force to dislodge the ectocervical cell samples therefrom into the solution. Further, the disposition of the cell removal member totally within the solution placed in the container (so that the solution covers the top of the cell removal member) and the presence of the alignment member at the top of the container interfere with visualization of the cell removal member, thereby rendering use of the apparatus difficult.

Accordingly, it is an object of the present invention to provide a collection apparatus for containing a solution useful for retrieval of cell samples both from a spatula having a collector on a leading end thereof and from a cytology brush having bristles on a leading end thereof.

Another object is to provide such apparatus which, in one embodiment, is useful for the collection of both endocervical and ectocervical cell samples.

A further object is to provide such apparatus which, in one embodiment, has an endocervical cell removal member which is asymmetrically disposed and only partially submerged within the solution and there is no alignment member at the open end of the container, thereby to facilitate visualization of the endocervical cell removal member and movement of the spatula.

It is also an object of the present invention to provide such apparatus which, in one embodiment, is economical and simple to manufacture and use.

### SUMMARY OF THE INVENTION

It has now been found that the above and related objects of the present invention are obtained in a collection apparatus for containing a solution useful for retrieval of cell samples both from a spatula having a collector on a leading end thereof and from a cytology brush having bristles on a leading end thereof. The collection apparatus comprises a container configured and dimensioned for containing a quantity of the solution and defining an open end, a closed end, and a sidewall connecting the ends. A cap is securable to the open end of the container to create a fluid-tight seal of the open end after the cytology brush and the spatula are removed from the container. An endocervical cell removal member is disposed within the container so as to be at least partially submerged within a solution to be contained therein. The endocervical cell removal member defines a plurality of surfaces against which the bristles of a cytology brush can be brought into contact to permit endocervical cell samples present on the bristles to be dislodged and suspended with the solution. The exterior surface of the endocervical cell removal member provides a surface against which the collector of a spatula can be brought into contact to permit ectocervical cell samples present on the collector to be dislodged and suspended within the solution.

In a preferred embodiment, the endocervical cell removal member is asymmetrically disposed within the container and is only partially submerged within the solution to facilitate visualization thereof. The endocervical cell removal member provides a plurality of essentially horizontal and a plurality of essentially vertical agitation surfaces against which the bristles of a cytology brush can be agitated. The exterior surface of the endocervical cell removal member provides a surface against which the collector of the spatula can be agitated to dislodge ectocervical cell samples present on the collector and suspend them within the solution.

Preferably the apparatus additionally including an ectocervical cell removal member defining abutment surfaces against which the collector of the spatula can be struck so as to dislodge the ectocervical cell samples present on the collector and thus suspend the ectocervical cell samples within the solution. More particularly, the ectocervical cell removal member has abutment surfaces including an exterior surface of the endocervical cell removal member and an interior surface of the container remotely spaced from the exterior surface against which the collector of the spatula can be brought into contact to permit the ectocervical cell samples present on the collector to be dislodged and suspended within the solution. The ectocervical cell removal member is at least partially, and preferably only partially, submerged within the solution and extends thereabove for visualization.

In another preferred embodiment, the container is cylindrical in shape, the closed end thereof is generally flat, and the endocervical cell removal member is spaced from the sidewall. The endocervical cell removal member comprises a plurality of fins affixed to an interior surface of the container. Each fin projects inwardly so that the fins meet in a spaced apart and opposed relationship and form a fin passageway therebetween, the fin passageway being capable of receiving the leading end of the cytology brush such that the leading end of the cytology brush is substantially centered and the bristles may be agitated against each fin as the cytology brush is rotated.

Preferably, each of the plurality of fins has a top edge, a side lateral edge, an outer edge, and a bottom end such that the side lateral edges act in combination to form the fin passageway between the inwardly projecting fins. The fin passageway is substantially uniform in cross section and has a central longitudinal axis that is horizontally offset from the central longitudinal axis of the container, whereby the fin passageway is capable of receiving the leading end of the cytology brush so that the bristles thereof can be agitated against both the top and side lateral edges of each fin as the cytology brush is rotated. Preferably the top edge of each fin is slightly curved, and the junctions of the fin top and side lateral edges are beveled to define a self-centering entry to the fin passageway.

The exterior surface of the endocervical cell removal member that can be struck by the collector of the spatula as a part of the abutment surface is formed by the fin outer edges. The plurality of fins is affixed to an interior surface of the closed end of the container.

### BRIEF DESCRIPTION OF THE DRAWING

The above and related objects, features and advantages of the present invention will be more fully understood by reference to the following detailed description of the presently preferred, albeit illustrative, embodiments of the present invention when taken in conjunction with the accompanying drawing wherein:

FIG. 1 is an exploded front elevational view of the collection apparatus according to the present invention as well as a spatula and a cytology brush for use therewith;

FIG. 2 is a vertical sectional view of the apparatus, to an enlarged scale, showing the spatula introduced therein;

FIG. 3 is a top plan sectional view taken along the lines 3-3 of FIG. 2, with the spatula being shown in phantom line in various positions useful for removal of ectocervical cell samples therefrom;

FIG. 4 is a vertical sectional view of the apparatus, to an enlarged scale, similar to FIG. 2, but showing the cytology brush inserted into the endocervical cell removal member and indicating the motion of the brush useful for the removal of endocervical cells samples therefrom; and

FIG. 5 is a top plan sectional view, similar to FIG. 3, but taken along the line 5-5 of FIG. 4.

### DETAILED DESCRIPTION OF THE PREFERRED EMBODIMENTS

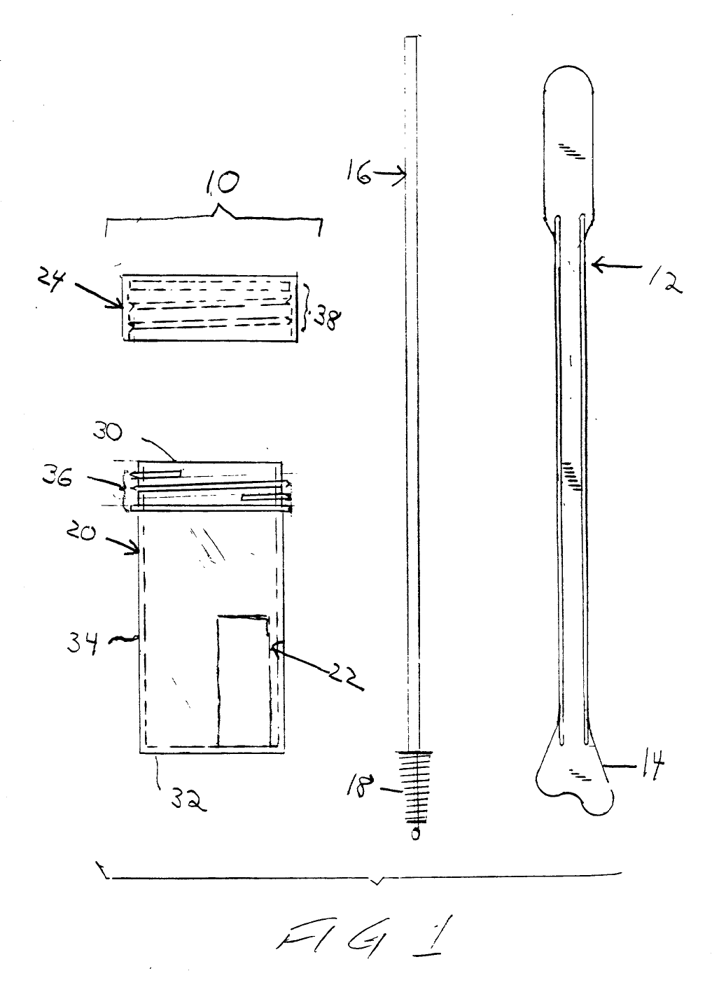

Referring now to the drawing, and in particular to FIG. 1 thereof, therein illustrated is collection apparatus according to the present invention, generally designated by the reference numeral 10, along with a spatula generally designated 12 having a collector 14 on a leading end thereof and a cytology brush device generally designated 16 having bristles 18 on a leading end thereof. As the spatula 12 and cytology brush device 16 are of conventional design and well-known to those skilled in the art of Pap smears, it is not deemed necessary to provide further details thereof herein. It will be appreciated that by such artisans that the collector 14 on the leading end of the spatula 12 is suitably configured, dimensioned and adapted for the collection of ectocervical cell samples from the exterior of a cervix, and that the brushes 18 on the leading end of the cytology brush device 16 are configured, dimensioned and adapted for the collection of endocervical cell samples from the interior of a cervix. The rounded tip of the leading end of the cytology brush device 16 is simply to preclude injury to the patient during endocervical cell collection.

The collection apparatus 10 comprises a container generally designated 20, an endocervical cell removal member generally designated 22 disposed within the container 20, and a cap generally designated 24 securable to the container 20.

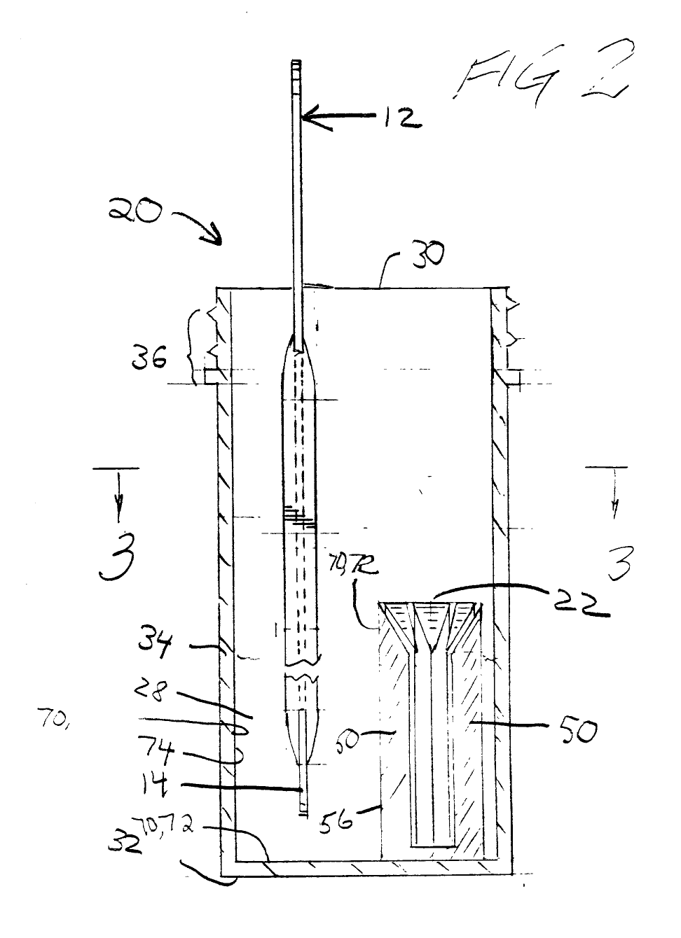

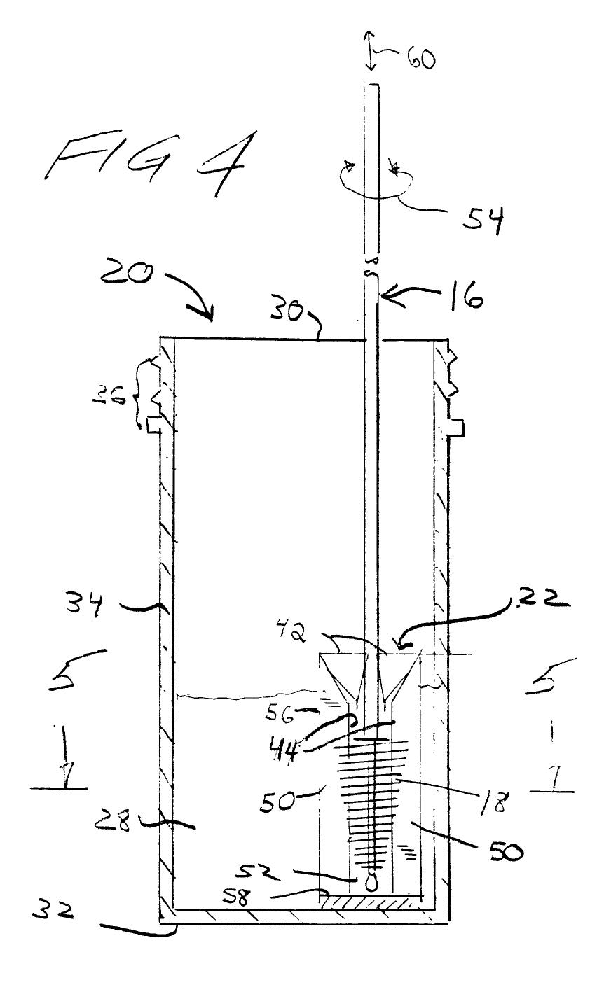

More particularly, the container 20 is configured and dimensioned for containing a quantity of the fixative solution 28 (see FIGS. 2 and 4) conventionally used for the fixing of collected cervical cell samples. (An example of a suitable fixative solution is disclosed in U.S. Pat. No. 5,256,571.) The container 20 is preferably cylindrical in configuration and defines an open end 30, a closed end 32 and a sidewall 34 connecting the ends 30, 32. The sidewall 34 is a hollow cylinder, and its outer surface is preferably externally threaded at 36 adjacent the open end 30.

The cap 24 is securable to the container open end 30 to create a fluid-tight seal of the open end 30 and is preferably internally threaded at 38. Of course, the external thread of container 20 at 36 and the internal thread of cap 24 at 38 are only examples of various means well-known the art to secure a cap to a container to create a fluid-tight seal of the open end, and other equivalent means well-known in the art may be used in the present invention. It will be appreciated that, absent cap 24, the container open end 30 is not blocked in any way and thus does not limit the introduction therethrough either of cytology brush 12 or spatula 16 or the free movement of the collector 14 or bristles 18 within container 20.

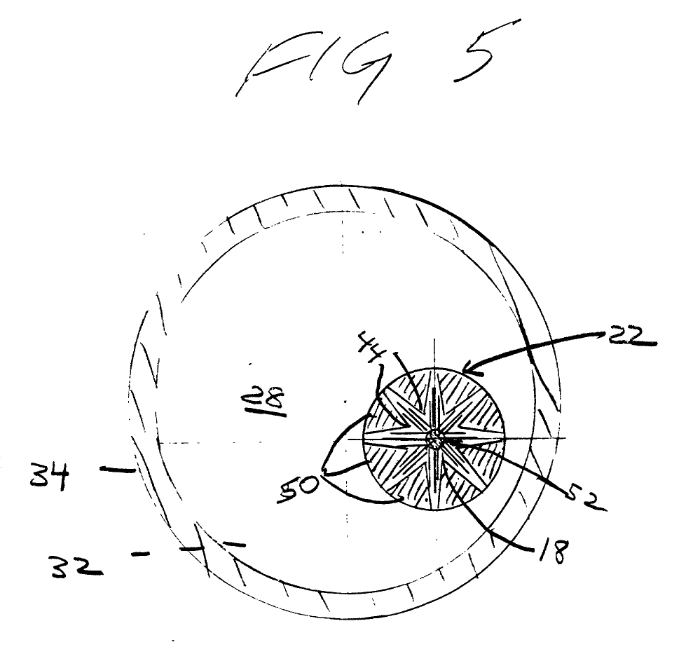

Referring now to FIGS. 4 and 5 in particular, therein illustrated is the open-top container 20 containing a quantity of solution 28 and a cytology brush 16 with its bristles 18 disposed within the endocervical cell removal member 22. The endocervical cell removal member 22 is disposed within the container 20 so as to be at least partially submerged within a solution 28 (such as the fixative solution) in the container 20. Preferably, the member 22 is only partially submerged within the solution 28 when the container 20 contains the appropriate quantity of the solution 28 so that the upper portion thereof is visible above the top of the solution, thereby to facilitate visualization of the member 22. For reasons which will become apparent hereinafter, the member 22 is asymmetrically disposed within the container 20—that is, it is offset from the central axis of the container 20 and adjacent (but preferably spaced from) the container sidewall 34. In this way, the exterior surface of member 22 provides a surface against which the collector 14 of spatula 12 can be agitated to dislodge ectocervical cell samples present on the collector 14 and suspend them within the solution 28.

The member 22 defines a plurality of surfaces against which the bristles 18 of a cytology brush 16 can be brought into contact to permit endocervical cell samples present on the bristles 18 to be dislodged and suspended within the solution 28. Thus, the member 22 provides a plurality of essentially horizontal agitation surfaces 42 and a plurality of essentially vertical agitation services 44, such agitation surfaces 42, 44 being provided to enable the cytology brush bristles 18 to be manually agitated by the user. More particularly, the member 22 comprises a plurality of fins 50 affixed to an interior surface of the container 30, and preferably to the interior surface of the container closed end 32. Each fin 50 projects inwardly so that the fins 50 meet in a spaced apart and opposed relationship about the central longitudinal axis of the member 22 and form a fin passageway 52 therebetween. The fin passageway 52 is capable of receiving the leading end of the cytology brush 16 such that the leading end is substantially centered and the bristles 18 thereof may be agitated against each fin 50 as the cytology brush 16 is rotated back and forth as indicated by the double headed arrow 54 of FIG. 4. Each of the fins 50 has a top edge 42, a side lateral edge 44, an outer lateral edge 56 and a bottom end 58. The side lateral edges 44 act in combination to form the fin passageway 52 between the inwardly projecting fins 50. The fin passageway 52 is substantially uniform in cross-section and has a central longitudinal axis (here, aligned with the cytology brush 16) that is horizontally offset from the central longitudinal axis of the container 20. The fin passageway 52 is capable of receiving the leading end of the cytology brush 16 so that the bristles 18 thereof can be agitated against both the top and side lateral edges 42, 44 of each fin 50 as the cytology brush 16 is rotated. To facilitate the self-centering entry of the cytology brush bristles 18 into the fin passageway 52, the top edge 42 of each fin 50 is slightly curved inwardly and downwardly and the junctions of the fin top and side lateral edges 42, 44 are beveled (whether linearly or curved) inwardly and downwardly to define a self-centering entry.

While the side lateral edges are illustrated as perpendicular to the container closed end 32 and parallel to the central longitudinal axis of the container 30, if desired the side lateral edges 44 beneath the self-centering entryway into the fin passageway 52 may be inclined downwardly and inwardly towards one another, thereby to provide increasing agitation towards the bottom for a cylindrical configuration of bristles and at least substantially constant agitation along the length of a conically configured disposition of bristles 18 (as illustrated).

As will be apparent, the rotational motion 54 applied to the cytology brush 16, optionally in combination with a reciprocating vertical movement (illustrated by the double headed arrow 60) provides adequate agitation to dislodge endocervical cell samples from the cytology brush bristles 18 and suspend them within solution 28.

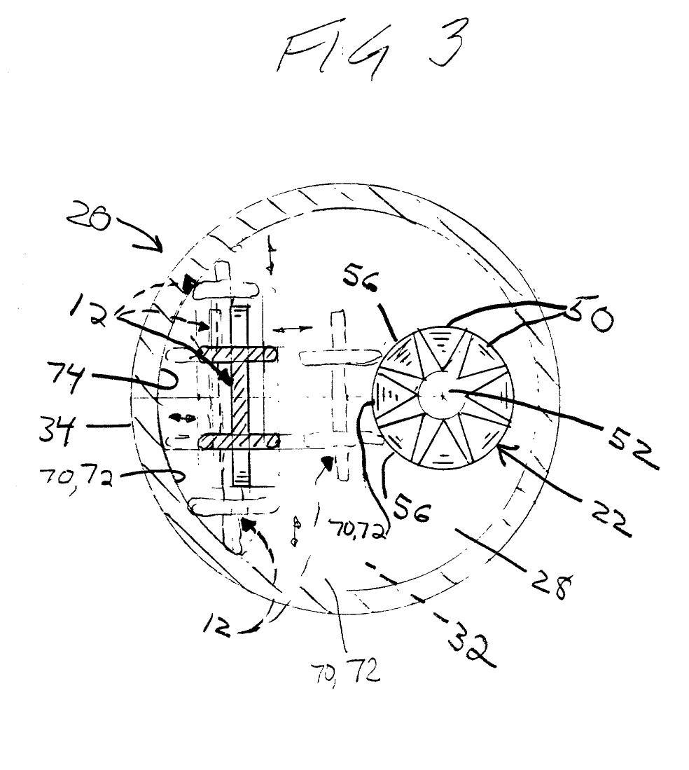

Referring now to FIGS. 2 and 3 in particular, therein illustrated is the open-top container 30 containing a quantity of solution 28 and a spatula 12 with its collector 14 disposed within the solution 28. The container 20 includes an ectocervical cell removal member, generally designated 70 which defines abutment surfaces 72 against which spatula collector 14 can be brought into contact so as to dislodge the ectocervical cell samples present on the collector 14 and thus suspend the ectocervical cell samples within the solution 28.

More particularly, the ectocervical cell removal member 70 is disposed within the container 20 so as to be at least partially submerged within the solution 28. The ectocervical cell removal member 70 has (and, in fact, is defined by) abutment surfaces 72. The abutment surfaces 72 include a portion of the exterior surface 56 of the endocervical cell removal member 22 (formed by the outer edges 56 of the fins 50) and a portion of the interior surface 74 of the container 20 remotely spaced from the exterior surface 56 of the endocervical cell removal member 22. The spatula collector 14 can be brought into contact with these abutment surfaces 72 to permit the ectocervical cell samples present on the collector 14 to be dislodged and suspended within the solution 28. Of course, the abutment surfaces 72 of the ectocervical cell removal member 70 also include a portion of the interior surface of the container closed end 32 remotely spaced from the endocervical cell removal member 22.

As illustrated in FIG. 3, spatula 12 may be moved by the user from an initial position (illustrated in solid line) to the various contact or striking positions (illustrated in phantom line) adjacent abutment surfaces 72, following the various double headed arrows of FIG. 3 as well as by other motions (e.g., swirling) effective to dislodge the ectocervical cell samples present on the collector 14 and suspended them within the solution 28. Reciprocating vertical movement of the spatula 12, which may include banging the collector 14 against the interior surface of the container closed end 32, may be employed to the same end function.

It will be appreciated that such lateral motions of the spatula 12 are made possible both by the asymmetrical disposition of the endocervical cell remover 22, which insures adequate space between the exterior surface 56 of the endocervical cell removal member 22 and the interior surface 74 of the container 20 remotely spaced from the fin outer edges 56, and by the essentially open nature of the container open end 30 which does not restrict the desired movement of the spatula 12 since it is devoid of any alignment member.

The present invention enables both endocervical and ectocervical cell samples to be collected in a single quantity of solution, with the collection process being easy, fast and thorough.

While the present invention has been described in the context of apparatus for retrieving cervical cell samples, clearly the principles thereof are equally applicable to the collection of other cell samples which must be collected both from a spatula having a collector and from a cytology brush having bristles.

To summarize, the present invention provides a collection apparatus for containing a solution useful for the retrieval of cell samples, both ectocervical cell samples from a spatula having a collector on a leading end of thereof and endocervical cell samples from a cytology brush having bristles on a leading end thereof. The endocervical cell removal member is only partially submerged within the solution and there is no alignment member at the open end of the container, thereby to facilitate visualization of the endocervical cell removal member and movement of the spatula. The apparatus is economical and simple to manufacture and use.

Now that the preferred embodiments have been shown and described in detail, various modifications and improvements thereon will become readily apparent to those skilled in the art. Accordingly, the spirit and scope of the present invention is to be construed broadly and limited only by the appended claims, and not by the foregoing specification.

1. A collection apparatus for containing a solution useful for retrieval of cell samples both from a spatula having a collector on a leading end thereof and from a cytology brush having bristles on a leading end thereof, the collection apparatus comprising:
(A) a container configured and dimensioned for containing a quantity of the solution and defining an open end, a closed end, and a sidewall connecting said ends;
(B) an endocervical cell removal member disposed within said container so as to be at least partially submerged within a solution to be contained therein, said endocervical cell removal member defining a plurality of surfaces against which the bristles of a cytology brush can be brought into contact to permit endocervical cell samples present on the bristles to be dislodged and suspended with the solution, the exterior surface of said endocervical cell removal member providing a surface against which the collector of a spatula can be brought into contact to permit ectocervical cell samples present on the collector to be dislodged and suspended within the solution; and
(C) a cap securable to said open end of said container to create a fluid-tight seal of said open end.

2. The apparatus of claim 1 wherein said endocervical cell removal member is asymmetrically disposed within said container.

3. The apparatus of claim 1 wherein said endocervical cell removal member is only partially submerged within the solution, thereby to facilitate visualization of the endocervical cell removal member.

4. The apparatus of claim 1 wherein the exterior surface of said endocervical cell removal member provides a surface against which the collector of the spatula can be agitated to dislodge ectocervical cell samples present on the collector and suspend them within the solution.

5. The apparatus of claim 1 wherein said endocervical cell removal member provides a plurality of essentially horizontal and a plurality of essentially vertical agitation surfaces against which the bristles of a cytology brush can be agitated.

6. The apparatus of claim 1 additionally including an ectocervical cell removal member defining abutment surfaces against which the collector of the spatula can be struck so as to dislodge the ectocervical cell samples present on the collector and thus suspend the ectocervical cell samples within the solution.

7. The apparatus of claim 1 additionally including an ectocervical cell removal member disposed within said container so as to be at least partially submerged within the solution, said ectocervical cell removal member having abutment surfaces including an exterior surface of said endocervical cell removal member and an interior surface of said container remotely spaced from said exterior surface against which the collector of the spatula can be brought into contact to permit the ectocervical cell samples present on the collector to be dislodged and suspended within the solution.

8. The apparatus of claim 1 wherein said container is cylindrical in shape, said closed end thereof is generally flat, and said endocervical cell removal member is spaced from said sidewall.

9. The apparatus of claim 1 wherein said endocervical cell removal member comprises:
a plurality of fins affixed to an interior surface of said container, each said fin projecting inwardly so that said fins meet in a spaced apart and opposed relationship and form a fin passageway therebetween, said fin passageway being capable of receiving the leading end of the cytology brush such that the leading end of the cytology brush is substantially centered and the bristles may be agitated against each said fin as the cytology brush is rotated.

10. The apparatus of claim 9 wherein each of said plurality of fins has a top edge, a side lateral edge, an outer edge, and a bottom end such that said side lateral edges act in combination to form said fin passageway between the inwardly projecting fins, and wherein said fin passageway is substantially uniform in cross section and has a central longitudinal axis that is horizontally offset from the central longitudinal axis of said container, whereby said fin passageway is capable of receiving the leading end of the cytology brush so that the bristles thereof can be agitated against both said fin top and side lateral edges as the cytology brush is rotated.

11. The apparatus of claim 10 wherein said top edge of each said fin is slightly curved.

12. The apparatus of claim 10 wherein the junctions of said fin top and side lateral edges are beveled to define a self-centering entry to said fin passageway.

13. The apparatus of claim 10 wherein the exterior surface of said endocervical cell removal member that can be struck by the collector of the spatula as a part of said abutment surface is formed by said fin outer edges.

14. The apparatus of claim 9 wherein said plurality of fins is affixed to an interior surface of said closed end of said container.

15. The apparatus of claim 6 wherein said ectocervical cell removal member is only partially submerged within the solution and extends thereabove for visualization.

16. A collection apparatus for containing a solution useful for retrieval of cell samples both from a spatula having a collector on a leading end thereof and from a cytology brush having bristles on a leading end thereof, the collection apparatus comprising:
(A) a container configured and dimensioned for containing a quantity of the solution and defining an open end, a closed end, and a sidewall connecting said ends;
(B) an endocervical cell removal member spaced from said sidewall and asymmetrically disposed within said container so as to be only partially submerged within the solution contained therein, said endocervical cell removal member providing a plurality of essentially horizontal and a plurality of essentially vertical agitation surfaces against which the bristles of a cytology brush can be agitated so as to dislodge the endocervical cell samples present on the bristles and thus suspend the endocervical cell samples within the solution;
said endocervical cell removal member including a plurality of fins affixed to an interior surface of said container, each of said plurality of fins having a top edge, a side lateral edge, an outer edge, and a bottom end, each said fin projecting inwardly so that said side lateral edges of said fins meet in a spaced apart and opposed relationship and act in combination to form a fin passageway between said inwardly projecting fins, said fin passageway being capable of receiving the leading end of the cytology brush such that the leading end of the cytology brush is substantially centered and the bristles may be agitated against each said fin as the cytology brush is rotated, the junctions of said fin top and side lateral edges being beveled to define a self-centering entry to said fin passageway;
said fin passageway being substantially uniform in cross section and having a central longitudinal axis that is horizontally offset from the central longitudinal axis of said container, whereby said fin passageway is capable of receiving the leading end of the cytology brush so that the bristles thereof can be agitated against both said fin top and side lateral edges as the cytology brush is rotated;
(C) an ectocervical cell removal member disposed within the container so as to be at least partially submerged within the solution contained therein, said endocervical cell removal member providing abutment surfaces against which the collector of a spatula can be struck so as to dislodge the ectocervical cell samples present on the collector and thus suspend the ectocervical cell samples within the solution, said abutment surfaces including an exterior surface of said endocervical cell removal member and an interior surface of said container remotely spaced from said endocervical cell removal means, the exterior surface of said endocervical cell removal member that can be struck by the collector of the spatula as a part of said abutment surface being formed by said fin outer edges; and
(D) a cap member securable to said open end of said container to create a fluid-tight seal of said open end after the cytology brush and the spatula are removed from said container.

17. A collection apparatus for containing a solution is useful for retrieval of cell samples both from a spatula having a collector on a leading end thereof and from a cytology brush having bristles on a leading end thereof, the collection apparatus comprising:
(A) a container configured and dimensioned for containing a quantity of the solution and defining an open end, a closed end, and a sidewall connecting said ends;
(B) an endocervical cell removal member asymmetrically disposed within said container so as to be at least partially submerged within the solution contained therein, said endocervical cell removal member providing a plurality of essentially horizontal and a plurality of essentially vertical agitation surfaces against which the bristles of a cytology brush can be agitated so as to dislodge the endocervical cell samples present on the bristles and thus suspend the endocervical cell samples within the solution;
(C) an ectocervical cell removal member disposed within said container so as to be at least partially submerged within the solution contained therein, said ectocervical cell removal member for providing abutment surfaces against which the collector of a spatula can be struck so as to dislodge the ectocervical cell samples present on the collector and thus suspend the ectocervical cell samples within the solution, said abutment surfaces including an exterior surface of said endocervical cell removal member and an interior surface of said container remotely spaced from said endocervical cell removal means; and
(D) a cap securable to said open end of said container for creating a fluid-tight seal after the cytology brush and the spatula are removed from said container.

18. The apparatus of claim 17 wherein said container is cylindrical in shape, said closed end thereof is generally flat, and said endocervical cell removed member is spaced from said sidewall.

19. The apparatus of claim 17 wherein said endocervical cell removal member comprises:
a plurality of fins affixed to an interior surface of said closed end of said container, each said fin projecting inwardly so that said fins meet in a spaced apart and opposed relationship and form a fin passageway therebetween, said fin passageway being capable of receiving the leading end of the cytology brush such that the leading end of the cytology brush is substantially centered and the bristles may be agitated against each said fin as the cytology brush is rotated.

20. The apparatus of claim 19 wherein each of said plurality of fins has a top edge, a side lateral edge, an outer edge, and a bottom end such that said side lateral edges act in combination to form said fin passageway between the inwardly projecting fins, and wherein said fin passageway is substantially uniform in cross section and has a central longitudinal axis that is horizontally offset from the central longitudinal axis of said container, whereby said fin passageway is capable of receiving the leading end of the cytology brush so that the bristles thereof can be agitated against both said fin top and side lateral edges as the cytology brush is rotated.

21. The apparatus of claim 20 wherein said top edge of each said fin is slightly curved.

22. The apparatus of claim 20 wherein the junctions of said fin top and side lateral edges are beveled to define a self-centering entry to said fin passageway.

23. The apparatus of claim 20 wherein the exterior surface of said endocervical cell removal member that can be struck by the collector of the spatula as a part of said abutment surface is formed by said fin outer edges.

24. The apparatus of claim 19 wherein said plurality of fins is affixed to an interior surface of said closed end of said container.

25. The apparatus of claim 17 wherein said endocervical cell removal member is only partially submerged within the solution and extends thereabove for visualization.

26. A collection apparatus for containing a solution useful for retrieval of cell samples both from a spatula having a collector on a leading end thereof and from a cytology brush having bristles on a leading end thereof, the collection apparatus comprising:
(A) a container configured and dimensioned for containing a quantity of the solution and defining an open end, a closed end, and a sidewall connecting said ends;
(B) an endocervical cell removal member spaced from said sidewall and asymmetrically disposed within said container so as to be only partially submerged within the solution contained therein, said endocervical cell removal member providing a plurality of essentially horizontal and a plurality of essentially vertical agitation surfaces against which the bristles of a cytology brush can be agitated so as to dislodge the endocervical cell samples present on the bristles and thus suspend the endocervical cell samples within the solution;
said endocervical cell removal member including a plurality of fins affixed to an interior surface of said closed end of said container, each of said plurality of fins having a top edge, a side lateral edge, an outer edge, and a bottom end, each said fin projecting inwardly so that said side lateral edges of said fins meet in a spaced apart and opposed relationship and act in combination to form a fin passageway between said inwardly projecting fins, said fin passageway being capable of receiving the leading end of the cytology brush such that the leading end of the cytology brush is substantially centered and the bristles may be agitated against each said fin as the cytology brush is rotated, the junctions of said fin top and side lateral edges being beveled to define a self-centering entry to said fin passageway;
said fin passageway being substantially uniform in cross section and having a central longitudinal axis that is horizontally offset from the central longitudinal axis of said container, whereby said fin passageway is capable of receiving the leading end of the cytology brush so that the bristles thereof can be agitated against both said fin top and side lateral edges as the cytology brush is rotated;
(C) an ectocervical cell removal member disposed within the container so as to be at least partially submerged within the solution contained therein, said ectocervical cell removal member providing abutment surfaces against which the collector of a spatula can be struck so as to dislodge the ectocervical cell samples present on the collector and thus suspend the ectocervical cell samples within the solution, said abutment surfaces including an exterior surface of said endocervical cell removal member and an interior surface of said container remotely spaced from said exterior surface, the exterior surface of said endocervical cell removal member that can be struck by the collector of the spatula as a part of said abutment surface being formed by said fin outer edges; and
(D) a cap securable to said open end of said container to create a fluid-tight seal after the cytology brush and the spatula are removed from said container.
